(19) United States
(12) Patent Application Publication
(10) Pub. No.: US 2017/0100174 A1
(43) Pub. Date: Apr. 13, 2017

(54) STRESS SHIELDING AND VARIABLE TENSIONING SYSTEM FOR PELVIC FRACTURE MANAGEMENT OF OSTEOPOROTIC BONES

(71) Applicant: EMPIRE TECHNOLOGY DEVELOPMENT LLC, Wilmington, DE (US)

(72) Inventors: HIMANSHU MISHRA, Bangalore (IN); MONA SHARMA, JABALPUR (IN)

(73) Assignee: EMPIRE TECHNOLOGY DEVELOPMENT LLC, Wilmington, DE (US)

(21) Appl. No.: 15/290,161

(22) Filed: Oct. 11, 2016

(30) Foreign Application Priority Data
Oct. 12, 2015 (IN) 3277/DEL/2015

(51) Int. Cl. A61B 17/80; A61B 17/74

(52) U.S. Cl. CPC A61B 17/8014; A61B 17/746; A61B 2017/564

(57) ABSTRACT

An apparatus for use with a medical device is disclosed that includes an end plate. The end plate includes a fixation portion configured to attach to a proximal bone segment. The end plate further includes a second portion including an opening configured to receive a medical device. The apparatus further includes a tightening head shaped for selective placement within the opening. The tightening head is further configured for engagement with the medical device.

### CROSS-REFERENCE TO RELATED APPLICATION

This application claims priority under 35 U.S.C. §119 to Indian Patent Application No. 3277/DEL/2015, filed on Oct. 12, 2015, which is incorporated herein by reference in its entirety.

### BACKGROUND

Internally fixated implants are often used in orthopedics to repair fractured bones. Examples of internally fixated implants include implants that are implanted into the body to secure a fractured bone so that the fracture can effectively heal. In other words, implants include devices that can support a damaged biological structure until it is healed. Implants may also be utilized to enhance existing biological structure in the body.

Implants used to help heal fractures in human bone may include pins, rods, and screws. When a fractured bone is held in place by an implant, the implant can help prevent the fracture from worsening. In other words, the implant can keep the fracture from getting larger and/or keep the two fractured portions of bone close together so that less bone needs to grow for the fracture to heal. Another function of implants may be to share loading with a fractured bone or completely absorb loading from a fractured bone so that the bone does not undergo the stresses it normally would, which can facilitate healing of a fracture and prevent further injury or damage.

Implants can be located on the outside or surface of a bone or may be implanted inside a bone. The location and shape of an implant may depend on the type of bone involved, the type and location of the fracture, and the severity and location of any accompanying injuries that occurred along with a fracture.

### SUMMARY

An illustrative apparatus includes an end plate. The end plate includes a fixation portion configured to attach to a proximal bone segment. The end plate further includes a second portion including an opening configured to receive a medical device. The apparatus further includes a tightening head shaped for selective placement within the opening. The tightening head is further configured for engagement with the medical device.

A method of compacting a bone fracture, the method includes inserting an adjustment portion of an end plate into a bone tunnel. The adjustment portion is configured to receive a medical device. The method further includes attaching a fixation portion of the end plate to a proximal bone segment. The method further includes inserting the medical device into the bone tunnel and the adjustment portion of the end plate such that a head portion of the medical device engages a tightening head in the adjustment portion. The method further includes adjusting the tightening head causing the medical device to be adjusted.

An illustrative system includes a bone implant and an end plate. The end plate includes a fixation portion configured to attach to a proximal bone segment. The end plate also includes a second portion including an opening configured to receive the bone implant. The system further includes a tightening head shaped for selective placement within the opening, wherein the tightening head is further configured for engagement with the bone implant, and further wherein the tightening head is configured to engage the end plate and the bone implant.

### BRIEF DESCRIPTION OF THE DRAWINGS

Illustrative embodiments will hereafter be described with reference to the accompanying drawings.

FIG. 1 depicts a perspective view of an end plate assembly and a medical device in accordance with an illustrative embodiment.

FIG. 2 depicts a perspective view of an end plate assembly and a medical device implanted into a fractured human pelvic bone in accordance with an illustrative embodiment.

FIG. 3A depicts a cross-sectional view of an end plate assembly and a medical device implanted into a fractured human pelvic bone in accordance with an illustrative embodiment.

FIG. 3B depicts a cross-sectional view of a medical device with no end plate assembly implanted into a fractured human pelvic bone in accordance with an illustrative embodiment.

FIG. 4A depicts a cross-sectional view of the tensioning force caused by an end plate assembly and a medical device implanted into a fractured human pelvic bone in accordance with an illustrative embodiment.

FIG. 4B depicts a cross-sectional view of the tensioning force caused by a medical device with no end plate assembly implanted into a fractured human pelvic bone in accordance with an illustrative embodiment.

FIG. 5A depicts a perspective view of a bore in a fractured human pelvic bone in accordance with an illustrative embodiment.

FIG. 5B depicts a cross-sectional view of a bore in a fractured human pelvic bone in accordance with an illustrative embodiment.

FIG. 6A depicts a perspective view of an end plate in a bore in a fractured human pelvic bone in accordance with an illustrative embodiment.

FIG. 6B depicts a cross-sectional view of an end plate in a bore in a fractured human pelvic bone in accordance with an illustrative embodiment.

FIG. 7A depicts a perspective view of fasteners and an end plate in a bore in a fractured human pelvic bone in accordance with an illustrative embodiment.

FIG. 7B depicts a cross-sectional view of fasteners inserted into an end plate and a proximal segment of human pelvic bone in accordance with an illustrative embodiment.

FIG. 8A depicts a perspective view of a medical device inserted into an end plate and a bore in a fractured human pelvic bone in accordance with an illustrative embodiment.

FIG. 8B depicts a cross-sectional view of a medical device inserted into an end plate and a bore in a fractured human pelvic bone in accordance with an illustrative embodiment.

FIG. 9A depicts a perspective view of a tensioning tool used to adjust a medical device in a fractured human pelvic bone in accordance with an illustrative embodiment.

FIG. 9B depicts a cross-sectional view of a tensioning tool used to adjust a medical device in a fractured human pelvic bone in accordance with an illustrative embodiment.

FIG. 10A depicts a perspective view of a medical device, end plate assembly, and locking head in accordance with an illustrative embodiment.

FIG. 10B depicts a cross-section view of a medical device, end plate assembly, and locking head in accordance with an illustrative embodiment.

FIG. 11 depicts a non-exploded and corresponding exploded view of an end plate assembly and medical device in accordance with an illustrative embodiment.

FIG. 12 depicts a perspective view of an end plate assembly in accordance with an illustrative embodiment.

FIG. 13A depicts a cross-sectional view of an end plate assembly in accordance with an illustrative embodiment.

FIG. 13B depicts a perspective cross-sectional view of an end plate assembly in accordance with an illustrative embodiment.

FIG. 14A depicts a perspective view of the adjustability of an end plate in accordance with an illustrative embodiment.

FIG. 14B depicts a top view of the adjustability of an end plate in accordance with an illustrative embodiment.

FIG. 14C depicts various shapes of pelvic contours in accordance with an illustrative embodiment.

FIG. 15A depicts a cross-sectional view of a stepped bore in a proximal section of a fractured human pelvic bone in accordance with an illustrative embodiment.

FIG. 15B depicts a perspective cross-sectional view of a stepped bore in a proximal section of a fractured human pelvic bone in accordance with an illustrative embodiment.

FIG. 16A depicts a cross-sectional view of an end plate assembly in a stepped bore in a proximal section of a fractured human pelvic bone in accordance with an illustrative embodiment.

FIG. 16B depicts a perspective cross-sectional view of an end plate assembly in a stepped bore in a proximal section of a fractured human pelvic bone in accordance with an illustrative embodiment.

FIG. 17A depicts a cross-sectional view of a medical device in an end plate assembly in a stepped bore in a proximal section of a fractured human pelvic bone in accordance with an illustrative embodiment.

FIG. 17B depicts a perspective cross-sectional view of a medical device in an end plate assembly in a stepped bore in a proximal section of a fractured human pelvic bone in accordance with an illustrative embodiment.

FIG. 18A depicts a cross-sectional view of a tensioning tool used to adjust a medical device in a fractured human pelvic bone in accordance with an illustrative embodiment.

FIG. 18B depicts a perspective cross-sectional view of a tensioning tool used to adjust a medical device in a fractured human pelvic bone in accordance with an illustrative embodiment.

FIG. 19A depicts a detail area of a cross-sectional view of a tensioning tool used to adjust a medical device in a fractured human pelvic bone in accordance with an illustrative embodiment.

FIG. 19B depicts an enlarged cross-sectional view of a tensioning tool used to adjust a medical device in a fractured human pelvic bone in accordance with an illustrative embodiment.

FIG. 20A depicts a cross-sectional view of a tensioning tool used to adjust a tightening head of an end plate assembly in accordance with an illustrative embodiment.

FIG. 20B depicts a perspective cross-sectional view of a tensioning tool used to adjust a tightening head of an end plate assembly in accordance with an illustrative embodiment.

FIG. 21A depicts a detail area of a cross-sectional view of a tensioning tool used to adjust a tightening head of an end plate assembly in accordance with an illustrative embodiment.

FIG. 21 B depicts an enlarged cross-sectional view of a tensioning tool used to adjust a tightening head of an end plate assembly in accordance with an illustrative embodiment.

FIG. 22A depicts a cross-sectional view of locking head in an end plate assembly in accordance with an illustrative embodiment.

FIG. 22B depicts a perspective cross-sectional view of locking head in an end plate assembly in accordance with an illustrative embodiment.

FIG. 23 is a flow diagram illustrating a method for inserting and adjusting a medical device in accordance with an illustrative embodiment.

FIG. 24 is a flow diagram illustrating a method for adjusting an end plate in accordance with an illustrative embodiment.

FIG. 25 is a flow diagram illustrating a method for simultaneously adjusting the radial expansion of a bone implant and pulling a distal and proximal bone segment together in accordance with an illustrative embodiment.

### DETAILED DESCRIPTION

Described herein are illustrative embodiments for methods and systems that provide for a stress shielding and variable tensioning system for fracture management. The systems and methods disclosed herein provide for an end plate that can be secured to a bone such as a pelvis. The end plate helps distribute stress acting on bone-implant interfaces across the surface of a bone where the end plate is attached. The end plate may also extend into a bore formed in a bone (also referred to herein as a bone tunnel). In this way, the end plate can interact or secure to an implant with a medical device implanted into the bone tunnel.

In an illustrative embodiment, the end plate may provide a system and method for variable adjusting and/or tensioning a medical device. In other words, the medical device can be adjusted and/or tensioned in multiple ways. For example, in an illustrative embodiment, a bone implant that is expandable can be inserted through a bore in a bone (e.g., a bone tunnel). Such a bone implant may be configured to expand radially into the bore and stiffen. In other words, the adjustable bone implant may change width in response to rotational movement of a head portion of the bone implant with respect to the rest of the bone implant. Such a bone implant can also be flexible, so that it can fit into different shapes of a bore (e.g., bone tunnel). The bore can pass from a proximal segment of bone across a fracture into a distal segment of bone on the other side of a fracture. In this way, the bone implant can be radially expanded within the bore in order to create tension with the bone on the walls of the bore. Further, such an implant may also be tensioned and/or stiffened to provide additional support to the bone around it. After the radial expansion, the proximal and distal segments of the bone are better held in place to allow for healing of the fracture.

In an illustrative embodiment, the bone implant can also be tensioned and/or adjusted in another way with the incorporation of an end plate as disclosed herein. For example, a bone implant that can radially expand as described above may be used in conjunction with the end plate. In an illustrative embodiment, a fixation portion of the end plate is configured to attach to a proximal bone segment of a fractured bone. A second portion of the end plate has an opening that is configured to receive an adjustable medical device, such as the radially expandable bone implant described herein. The second portion may be inserted into the bore in the bone (otherwise referred to as the bone tunnel). In order to accommodate a medical device and the end plate in the bone tunnel, the bone tunnel may be stepped such that a larger width part of the bone tunnel can accommodate the end plate and the stepped down smaller width part of the bone tunnel accommodates the medical device (which here is the bone implant). The end plate can also include a tightening head that is shaped for selective placement within the opening of the end plate. The tightening head is also configured for engagement with the medical device. In other words, the tightening head provides for adjusting and/or tensioning the medical device. In this example, the tightening head can pull a bone implant that is anchored in a distal bone segment such that the distal bone segment and the proximal bone segment are pulled together and/or compressed. In other words, the bone implant can be adjusted and/or tensioned to compact a fracture in a bone, as well as radially expanded to secure the implant and bone segments in place to facilitate healing.

The tightening head itself may be shaped in different ways. In an illustrative embodiment, the tightening head may be formed as a separate piece to an end plate. The tightening head may include an outer portion that is threaded to screw into and/or interact with threads in the opening of the end plate. In other words, the tightening head can be turned rotationally to move the tightening head up and down with respect to the end plate and in the opening of the end plate. An inner portion of the tightening head has an opening or bore of varying sizes and/or shapes to match the corresponding implant head. In an illustrative embodiment, the inner portion of the tightening head has three different bores. All three bores are different sizes. The first bore is the largest of the three and is shaped to let a medical device such as the bone implant from above pass through. The first bore is also shaped to receive a tensioning tool, such that the tightening head can be rotated as described above. The tightening head therefore can move up and down within the end plate using a tensioning tool that fits into the first bore. The second bore of the tightening head is shaped to be smaller in size than the first bore, and is shaped to receive a head portion of a medical device such as the bone plant described above. When inserting the bone implant through the tightening head, an extended portion (that extends from the head portion) can be inserted through the first and second bores. The head portion can rest in the second bore. The third bore at the base of the tightening head is shaped so that most of an extended portion of the bone implant can pass through the third bore, but it is shaped to such that the head portion of the bone implant can be held in the second bore without passing into the third bore. For example, the third bore may have a smaller diameter than the second bore. In this way, the third bore can ensure that the head portion of the bone implant stays in the second bore. Accordingly, when the tightening head is rotated within the end plate (moving the tightening head up or down), the bone implant can move along with the tightening head. In this way, the additional mode of tensioning and/or adjusting the bone implant is realized. This occurs because the end of the extended portion of the bone implant opposite the head portion is configured to be anchored to a distal bone segment on the other side from the tightening head and end plate from the fracture, thus compressing the distal and proximal bone sections on either side of the fracture together.

In an illustrative embodiment, the fixation portion of the end plate includes a plurality of arms that can attach and/or anchor to the proximal bone segment. The plurality of arms may make up the fixation portion completely, or may be attached to the fixation portion of the end plate. The fixation portion can extend from an outer wall of the second portion of the end plate that has the opening. When the fixation portion is just the plurality of arms, the plurality of arms extend from the outer wall of the second portion of the end plate. The plurality of arms can also be adjusted to fit the contours of the bone. This can help distribute stress across the bone more evenly and adequately, helping to prevent future injury to the bone. For example, the plurality of arms may be movable/bendable in at least two directions and/or dimensions with respect to the second portion of the end plate. The plurality of arms may also have threaded bores that can receive a fastener such as a screw. In this way, fasteners may be used to anchor the end plate to the bone. In alternative embodiments, other fastening systems and methods may be used. As noted above, the second portion of the end plate with the opening is inserted into a bore (e.g., bone tunnel) in the bone itself. However, in an illustrative embodiment, the plurality of arms and the fixation portion fits onto the surface of the bone outside the bore. In an alternative embodiment, the end plate may be anchored to the bone inside the bore (e.g., bone tunnel)and still be configured for the variable tensioning disclosed herein. In this embodiment, the end plate may not have a plurality of arms.

An illustrative embodiment can also include a locking head. The locking head can include a threaded portion that also engages the threads in the opening of the end plate. The locking head can therefore be screwed into the opening of the end plate. When the locking head is tightened down onto the bone implant and/or the tightening head, the locking head can secure the bone implant and tightening head from moving. A non-threaded portion of the locking head may be shaped to fit into the first bore of the tightening head to ensure that the tightening head and bone implant do not move.

The first, second, and third bores of the tightening head may be varying shapes. For example, the first bore may be a hexagonal stepped bore to accommodate a tensioning tool. As another example, the second and third bores may be a cylindrical bore, with a step in between the two. In this way, the head portion of a bone implant would be able to freely rotate with respect to the tightening head (or in the converse the tightening head would be able to rotate freely with respect to the bone implant). Together, the first, second, and third bores may form a hollow cavity in the tightening head, allowing the bone implant to be inserted therein.

As discussed herein, an illustrative embodiment provides for variable tensioning of both the width of a bone implant, as well as the compression of a proximal and distal bone segments that are separated by a fracture. These tensions may be adjusted in various ways. For example, a tensioning tool may be inserted into the adjustment portion (or head portion) of the bone implant in order to radially expand (or generally change the width of) the bone implant. This provides better contact between the bone implant and the bone within the bone tunnel or bore in the bone. A different tensioning tool may be used to adjust the tightening head in a way that compresses the bone segments on either side of the fracture. In another embodiment, a tensioning tool can be used that adjusts the tightening head and the bone implant width at the same time. In this way, a more precise tensioning can be achieved using these various tools. This may be important especially for large fractures or for osteoporotic or otherwise weakened bones, because the level of customizability of the adjustments may be exploited to cater to the exact condition and strength of the bone.

Accordingly, the end plates disclosed herein are adaptable to medical devices such as a flexible pelvic repair implant. The methods and systems disclosed herein provide for tensioning and distributing induced stresses for a flexible pelvic repair implant. Such an implant can be fixated via an intra-bone tunnel formed in the cancellous section of the fractured segment of bone.

The methods and systems disclosed herein can be particularly advantageous for the management of fractures in osteoporotic bones. For example, tensioning and/or adjustment of internal fixation devices as disclosed herein reduces the risk of implant failure by improving stability, making primary bone healing with minimal callus formation possible. The tensioning and/or adjustment as disclosed herein also enables controlled bone impaction, which is key for timely healing of fractured bone surfaces. The tensioning and/or adjustment as disclosed herein also can be used to produce final precise reduction and compression after approximate anatomic reduction.

The embodiments disclosed herein also advantageously provide for distribution of tension forces such that undue stress concentration is prevented at a bone-implant interface. Moreover, for osteoporotic bones, stress concentration may increase due to structural deterioration of bone architecture and a general decrease in bone density. Such stress concentration can lead to bone failure and loosening of an implant due to microfracture and bone resorption. Such bone failure is a common mode of failure of internal fixation in osteoporotic bones.

Further, the extent of tensioning and bone compaction for fracture fixation is not the same for all osteoporotic bones. The tensioning and bone compaction used to treat a fracture can be determined by many factors, including the degree of osteoporosis present, the desired anatomical reduction, and the location of the fracture. Thus, the embodiments disclosed herein advantageously address these issues and provide for systems and methods that provide better distribution of tension forces throughout an osteoporotic bone and allow for more variability of tensioning to adjust for important factors considered when treating a fracture in an osteoporotic bone. In other words, the embodiments disclosed herein advantageously include a flexible implant design suited for osteoporotic bones and provides for variable implant tensioning and preventing undue stress concentration.

Accordingly, disclosed herein is a stress shielding and variable tensioning system designed to be used with a flexible implant for osteoporotic pelvic fractures. An illustrative embodiment is an end plate construction positioned at the proximal end of a medical device such as a flexible implant.

The issues with treating a fractured osteoporotic bone are addressed by providing an end plate that causes wide buttressing of forces. The end plate spreads forces over a larger surface area of stronger cortical bone, thus preventing undue stress concentration at the weaker cancellous bone-implant interface. Further, the issues of treating a fractured osteoporotic bone are addressed through variable tensioning. An end plate can use a thread based embodiment to allow variable tensioning. In this way, a surgeon may exercise control over the extent of tensioning and bone compaction independently, based on the bone state and fracture specifics. Further, this embodiment allows both tensioning and actuation of a medical device like a flexible implant simultaneously. In this embodiment actuation of the flexible implant would be the radial bead expansion and stiffening of the flexible implant.

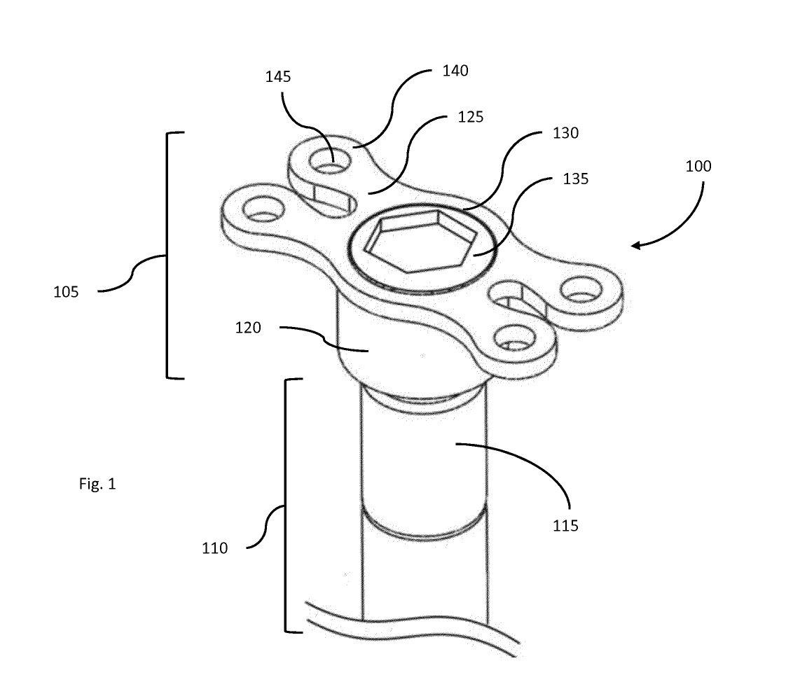

FIG. 1 depicts a perspective view of an apparatus 100 that includes an end plate assembly 105 and a medical device 110 in accordance with an illustrative embodiment. In alternative embodiments, fewer, additional, and/or different components may be included in the system. In this embodiment, the medial device 110 is a flexible bone implant. An extended portion 115 of the medical device 110 is visible in FIG. 1.

The end plate assembly 105, also referred to herein as a stress shielding and variable tensioning end plate, includes an end plate 125. The end plate 125 includes a fixation portion 140 and a second portion 120 that includes an opening 130. The opening 130 is configured to receive and house a (not visible) head portion of the medical device 115. In FIG. 1, a locking head 135 is shown within the opening 130. The locking head 135 and other aspects of the opening 130 are discussed at length below. The fixation portion 140 includes a bore 145. The bore 145 is able to receive a fastener such as a screw. In this way, the fixation portion 140 may be fixated to a bone.

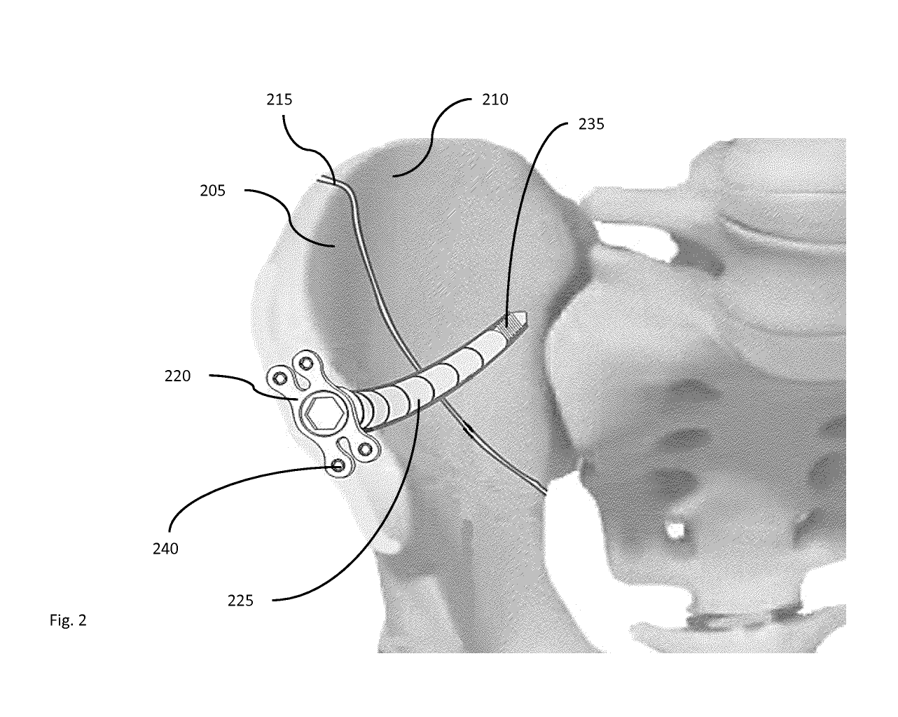

FIG. 2 depicts a perspective view of an end plate assembly 220 and a medical device 225 implanted into a fractured human pelvic bone in accordance with an illustrative embodiment. In alternative embodiments, fewer, additional, and/or different components may be included in the system. FIG. 2 demonstrates the end plate assembly 220 and the medical device 225 in an implanted state.

A fixation portion 240 of the end plate assembly 220 is fixated to a proximal bone segment 205. A distal bone segment 210 is on the other side of a fracture 215. The medical device 225 includes a threaded portion 235. The threaded portion 235 can be used to anchor or fixate the medical device 225 to the distal bone segment 210.

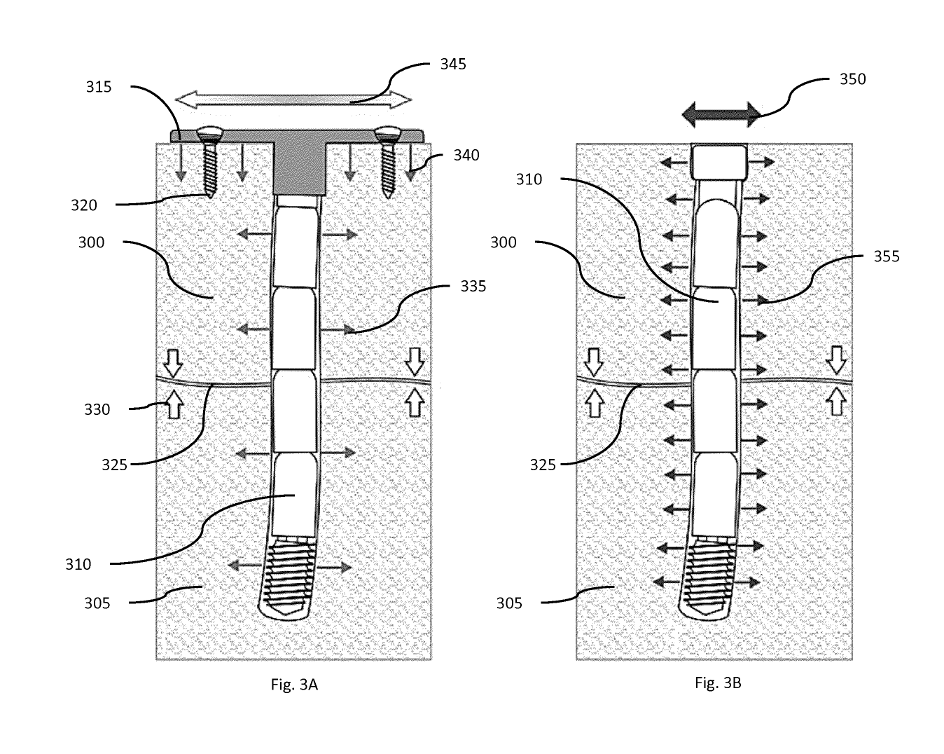

FIG. 3A depicts a cross-sectional view of an end plate assembly 315 and a medical device 310 implanted into a fractured human pelvic bone in accordance with an illustrative embodiment. In alternative embodiments, fewer, additional, and/or different components may be included in the system. FIG. 3A shows a distal bone segment 305 and a proximal bone segment 300. Between the two segments is shown a fracture 325. A medical device 310 is implanted into the bone across the fracture 325 and through parts of both the proximal bone segment 300 and the distal bone segment 305. FIG. 3A also depicts an end plate assembly 315 that is fixated to the proximal bone segment 300 with a fastener 320.

In FIG. 3A, the use of an end plate assembly increases the stress distribution (i.e., the stress is distributed across a larger area) on the proximal bone segment 300 and the distal bone segment 325. For example, an increased stress distribution across a larger area is shown by arrow 345. Arrows 335 and 340 also show relatively low stress because stress is shared by the end plate assembly 315. Arrow 330 shows the compression force that the end plate assembly 315 and the medical device 310 can exert on the proximal bone segment 300 and the distal bone segment 305 to pull the two segments together and/or compress the two segments at the fracture 325.

The end plate assembly 315 adopts the principle of wide buttressing for stress distribution. This spreads the load over a larger surface area of stronger cortical bone (bone touching the fixation portion of the end plate assembly 315), sharing the load which would otherwise be acting only at weaker cancellous bone and implant interface. This embodiment is particularly effective for an osteoporotic bone because it avoids high strain at any single implant component while the medical device (here, an implant) maintains a large contact area at the bone-implant interface (the cancellous bone), thereby reducing strain.

FIG. 3B depicts a cross-sectional view of a medical device 310 with no end plate assembly implanted into a fractured human pelvic bone in accordance with an illustrative embodiment. In alternative embodiments, fewer, additional, and/or different components may be included in the system. In contrast to FIG. 3A, there is a medical device 310 implanted into a distal bone segment 305 and a proximal bone segment 300.

An arrow 350 demonstrates that the stress distribution in this embodiment is not as large as in FIG. 3A, and further is only at the weaker cancellous bone-implant interface. Further, arrow 355 shows that the stresses on that bone-implant interface are higher than the stress on the bone-implant interface in FIG. 3A. Accordingly, the end plate assembly 315 shown in FIG. 3A helps distribute stress across a larger part of bone than using the medical device 310 without the end plate assembly 315 as shown in FIG. 3B.

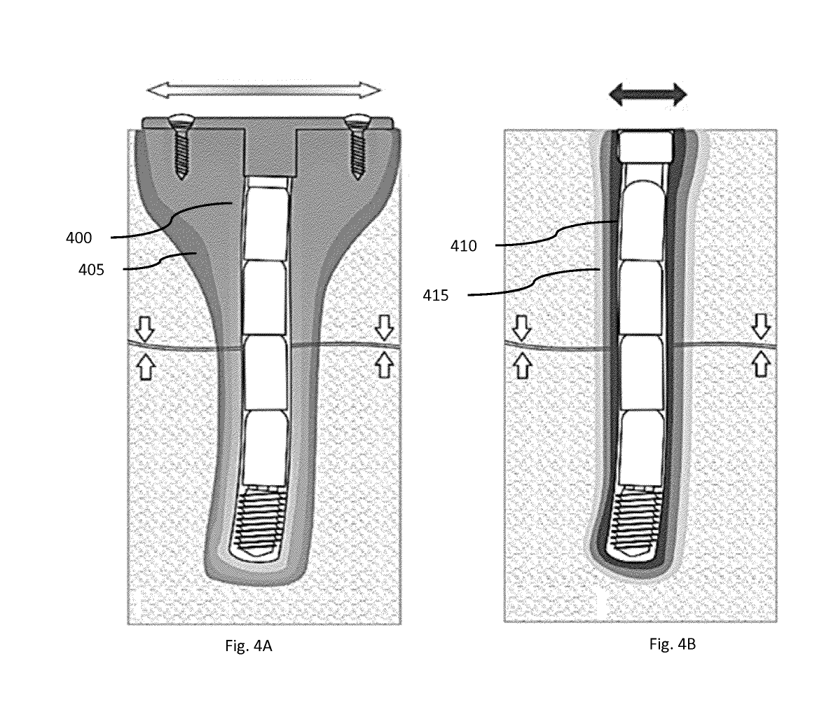

FIG. 4A depicts a cross-sectional view of the tensioning force caused by an end plate assembly and a medical device implanted into a fractured human pelvic bone in accordance with an illustrative embodiment. FIG. 4B depicts a cross-sectional view of the tensioning force caused by a medical device with no end plate assembly implanted into a fractured human pelvic bone in accordance with an illustrative embodiment. Similar to FIGS. 3A and 3B discussed above, FIGS. 4A and 4B show a medical device implanted into a fractured bone with (FIG. 4A) and without (FIG. 4B) an end plate assembly incorporated. FIG. 4A shows that tensioning forces are distributed over a larger surface area of intact bone than FIG. 4B (see tension shaded areas 400 and 405 in FIG. 4A versus 410 and 415 in FIG. 4B). By distributing tension forces over a larger surface area of intact bone, it also reduces the stress and tension levels at the fracture. Accordingly, this reduces the likelihood of complications or further injuries to a bone if the configuration in FIG. 4A is used as disclosed herein. This is particularly advantageous for weaker bones, for example osteoporotic bones.

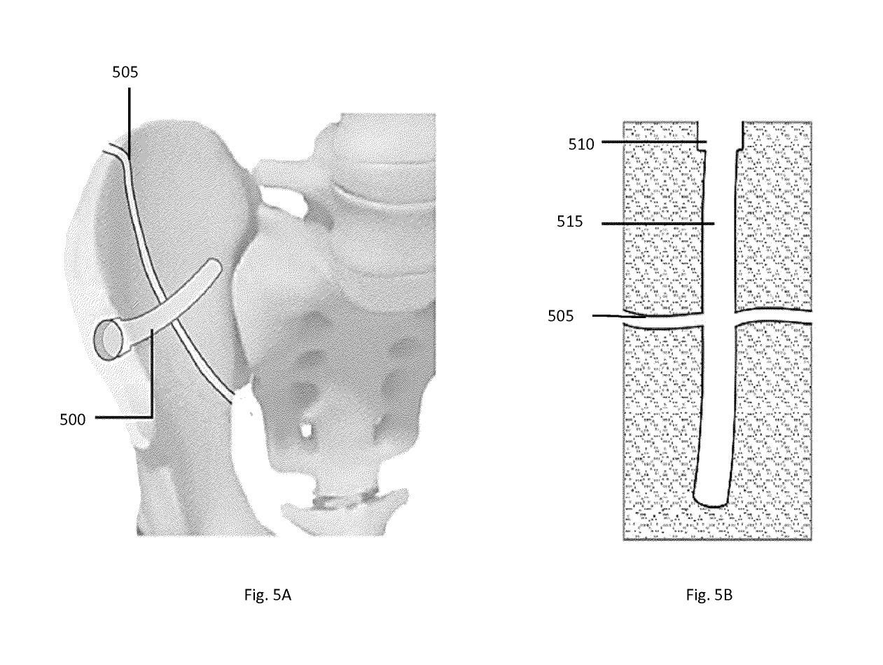

FIG. 5A depicts a perspective view of a bore 500 (also referred to herein as a bone tunnel) in a fractured human pelvic bone in accordance with an illustrative embodiment. In alternative embodiments, fewer, additional, and/or different components may be included in the system. In a first step for an illustrative embodiment, a bore 500 (or tunnel) is reamed in the pelvic bone. The bore 500 extends across a fracture 505 in the pelvic bone. Although a specific location and type of fracture is shown in FIG. 5A, the fracture may be shaped differently, located differently, or may be a different type of fracture in various alternative embodiments.

FIG. 5B depicts a cross-sectional view of a bore in a fractured human pelvic bone in accordance with an illustrative embodiment. In alternative embodiments, fewer, additional, and/or different components may be included in the system. In FIG. 5B, the shape of the bore 500 in FIG. 5A is more easily seen. The bore 500 of FIG. 5A includes a bone tunnel 515 and a step 510. The step 510 is at the proximal end and in the proximal bone segment. As will be disclosed in detail below, the bone tunnel 515 and step 510 of the bore 500 is configured to accommodate a medical device and an end plate.

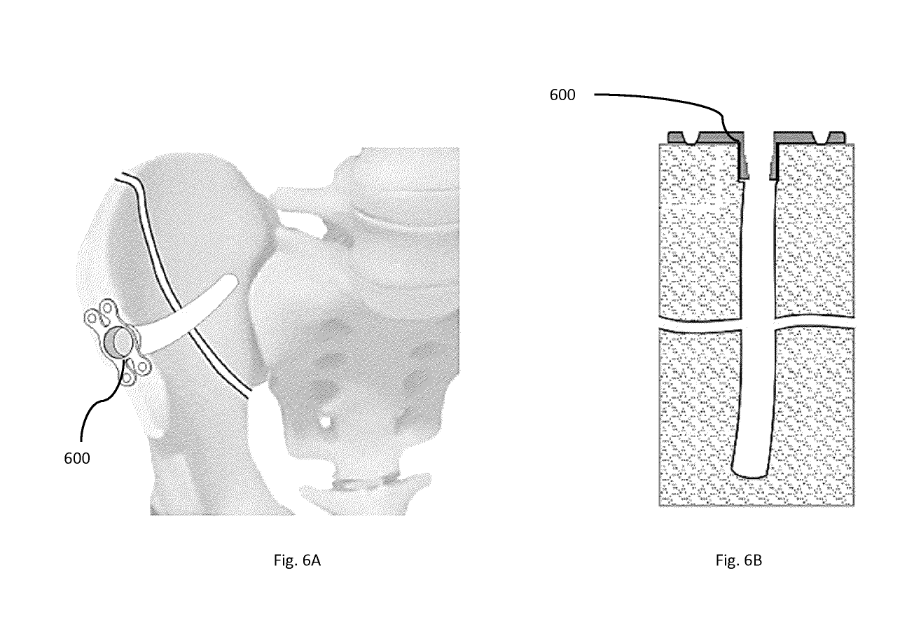

FIG. 6A depicts a perspective view of an end plate 600 in a bore in a fractured human pelvic bone in accordance with an illustrative embodiment. In alternative embodiments, fewer, additional, and/or different components may be included in the system. As shown in FIG. 6A, the end plate 600 is inserted at the proximal end of the bore in the pelvic bone.

FIG. 6B depicts a cross-sectional view of an end plate 600 in a bore in a fractured human pelvic bone in accordance with an illustrative embodiment. In alternative embodiments, fewer, additional, and/or different components may be included in the system. FIG. 6B also shows the end plate 600 inserted into the bore in the pelvic bone. Note that the fixation portion of the end plate stays outside of the bore, and the second portion of the end plate than includes an opening is inserted into the stepped portion of the bore in the pelvic bone. The end plate 600 in this embodiment is fixed to the proximal segment of bone through an interference fit. In other words, the stepped hole in the pelvic bone is shaped such that the second portion with the opening of the end plate 600 fits snugly into the stepped hole.

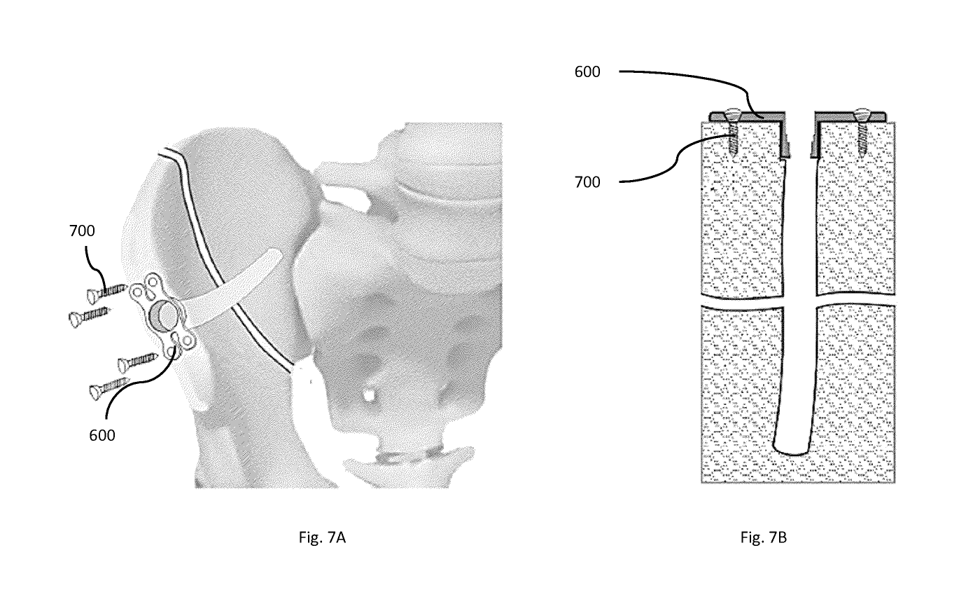

FIG. 7A depicts a perspective view of fasteners 700 and an end plate 600 in a bore in a fractured human pelvic bone in accordance with an illustrative embodiment. In alternative embodiments, fewer, additional, and/or different components may be included in the system. The fasteners 700 are shown not yet inserted into the pelvic bone or the end plate 600. When inserted into the end plate 600 and the pelvic bone, the fasteners 700 will secure or fixate the end plate to the pelvic bone.

FIG. 7B depicts a cross-sectional view of fasteners 700 inserted into an end plate 600 and a proximal segment of human pelvic bone in accordance with an illustrative embodiment. In alternative embodiments, fewer, additional, and/or different components may be included in the system. FIG. 7B shows the fasteners 700 actually inserted into the pelvic bone and bores in the fixation portion of the end plate 600. Here, the fasteners 700 are plate locking cortex screws. In FIG. 7B, the end plate 600 is fastened to the outer surface of the proximal segment of bone of the pelvis. In other alternative embodiments, the pelvis may be shaped or otherwise bored to accommodate the fixation portion of the end plate 600 such that the end plate and pelvic bone are essentially flush with each other. In this embodiment, the fixation portion of the end plate would not be attached to the surface of the pelvis as shown in FIG. 7B. In further alternative embodiments, fasteners other than the screws shown in FIGS. 7A and 7B may be utilized. For example, adhesives, rivets, pins, or other fasteners may be used to attach the end plate 600 to the pelvis (or other bone) surface.

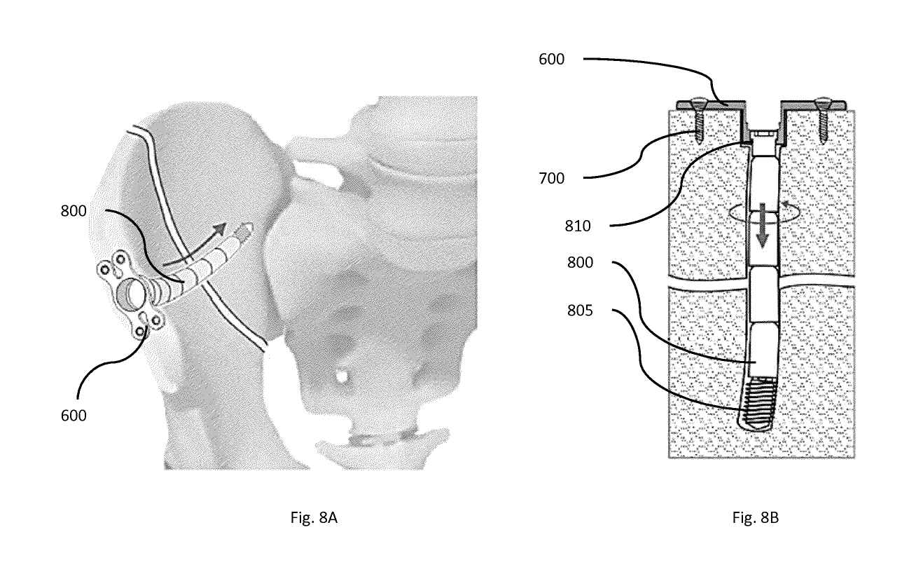

FIG. 8A depicts a perspective view of a medical device 800 inserted into an end plate 600 and a bore in a fractured human pelvic bone in accordance with an illustrative embodiment. In alternative embodiments, fewer, additional, and/or different components may be included in the system. FIG. 8A shows that the medical device 800, here a flexible implant, is inserted into the bone tunnel (or bore) and through the end plate 600. The medical device 800 extends the length of the bone tunnel and crosses the fracture, as well as extending through the proximal segment of the bone and into the distal segment of the bone. The medical device also extends through an opening (or bore) in the second portion of the end plate 600.

FIG. 8B depicts a cross-sectional view of a medical device 800 inserted into an end plate 600 and a bore in a fractured human pelvic bone in accordance with an illustrative embodiment. In alternative embodiments, fewer, additional, and/or different components may be included in the system. FIG. 8B shows another view of the medical device 800, here a flexible implant, inserted into the bone tunnel (or bore). The medical device 800 has a head portion 810 and a tail portion 805 that is threaded. During insertion, the medical device 800 can be rotated in order to anchor the medical device to the distal bone segment using the threads on the tail portion 805. In this embodiment, detail of a tightening head that is separate from the end plate 600 cannot be seen, but the head portion 810 of the medical device is held in place after insertion by a tightening head in the end plate 600. As disclosed herein, the tightening head can adjust the medical device 800 to pull the distal bone segment and proximal bone segment together (that is, to compress the fracture in the bone).

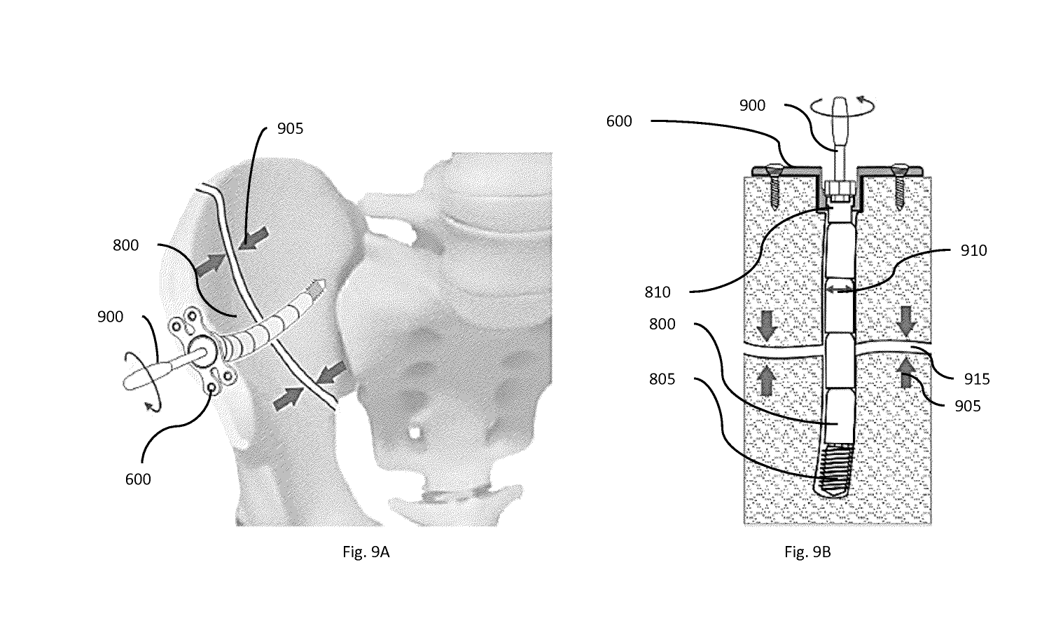

FIG. 9A depicts a perspective view of a tensioning tool 900 used to adjust a medical device 600 in a fractured human pelvic bone in accordance with an illustrative embodiment. In alternative embodiments, fewer, additional, and/or different components may be included in the system. The tensioning tool 900 used here is configured to tension the medical device 600 pulling (compressing) the distal and proximal bone segments together, while simultaneously actuating the implant (which stiffens the flexible implant and causes the beads of the flexible implant to expand in width, or expand radially). Arrow 905 shows the fracture being pulled together/compressed. In other words, the tensioning of the medical device 600 by rotating the tensioning tool 900 pulls the distal and proximal segments together (or compresses them against each other).

FIG. 9B depicts a cross-sectional view of a tensioning tool 900 used to adjust a medical device 600 in a fractured human pelvic bone in accordance with an illustrative embodiment. In alternative embodiments, fewer, additional, and/or different components may be included in the system. FIG. 9B demonstrates how the adjustment of the medical device 600 with the tensioning tool 900 causes actuation of the medical device 600 as well as tensioning of the medical device 600 simultaneously.

Arrow 905 shows how the two segments of bone (proximal and distal) are compressed against each other at the fracture 915. Arrow 910 shows how, when the tensioning tool 900 is rotated, the medical device 600 can radially expand (or increase in width) within the bone tunnel/bore in the human pelvic bone. The actuation of the medical device 600 not only expands the medical device 600, but also stiffens it as well.

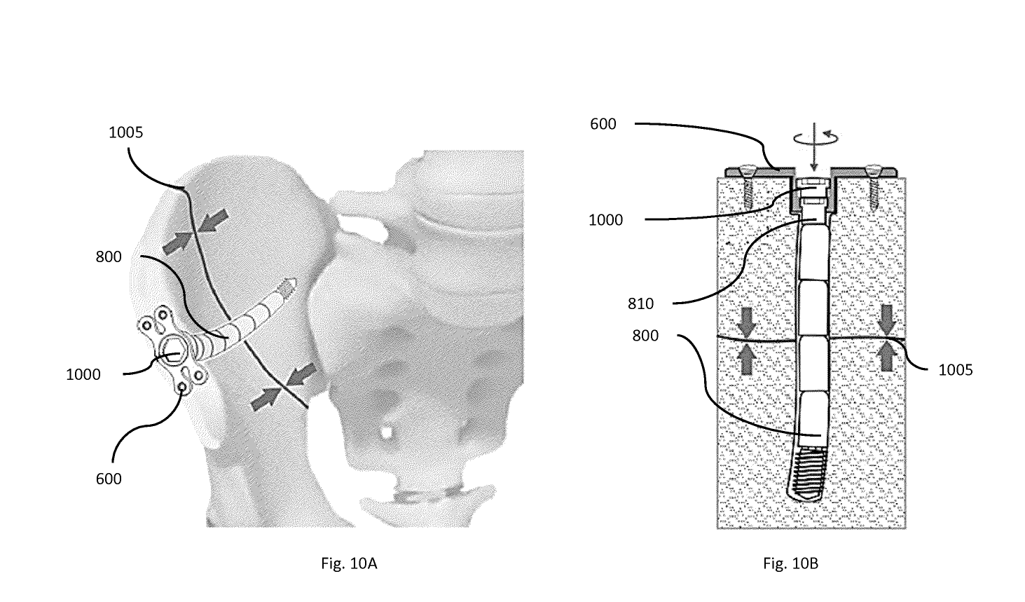

FIG. 10A depicts a perspective view of a medical device 800, end plate assembly 600, and locking head 1000 in accordance with an illustrative embodiment. In alternative embodiments, fewer, additional, and/or different components may be included in the system. FIG. 10B depicts a cross-section view of a medical device 800, end plate assembly 600, and locking head 1000 in accordance with an illustrative embodiment. In alternative embodiments, fewer, additional, and/or different components may be included in the system.

The locking head 1000 is inserted into the end plate assembly 1000. The locking head 1000 engages with the opening (bore) in the second portion of the end plate. The locking head 1000 can screw into the opening because both the outside of the locking head 1000 and the inside of the opening are correspondingly threaded. The locking head 1000 can be inserted into the end plate assembly 600 and fastened to the end plate in order to prevent back out of the flexible implant during ambulatory loading. As shown in FIGS. 10A and 10B, the locking head 1000 can be inserted after the medical device 800 has been tensioned and the proximal and distal bone segments have been compressed at the fracture 1005. The locking head 1000 can also prevent the head portion 810 of the medical device 800 from rotating, which could cause the medical device 800 to de-actuate or lose tension.

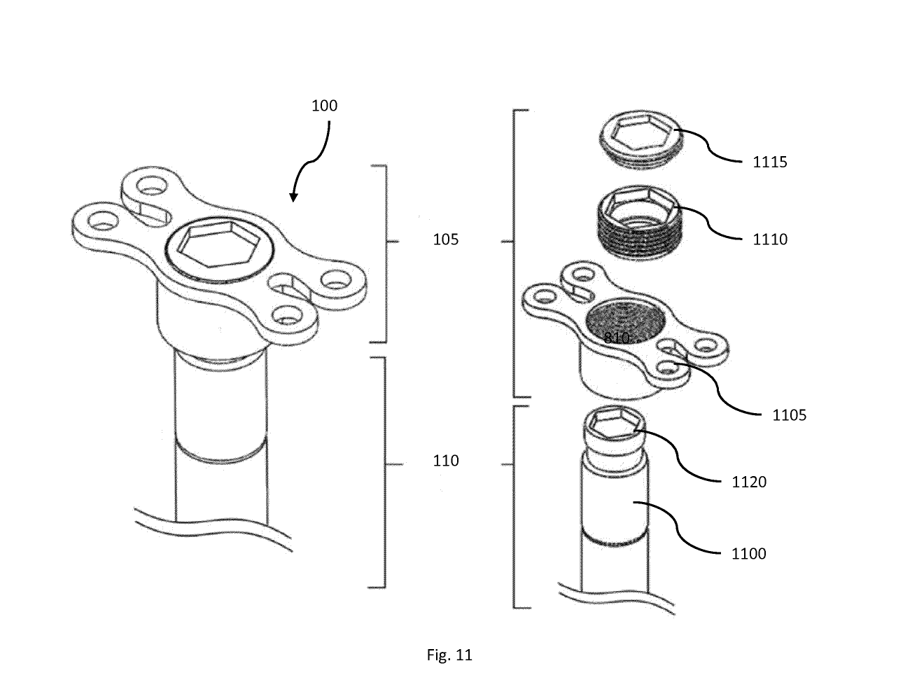

FIG. 11 depicts a non-exploded and corresponding exploded view of an end plate assembly 105 and medical device 110 in accordance with an illustrative embodiment. In alternative embodiments, fewer, additional, and/or different components may be included in the system. FIG. 11 shows the apparatus 100 including a medical device 110 and an end plate assembly 105 as shown in FIG. 1 and discussed above. In addition, FIG. 11 shows an exploded view of the components apparatus 100.

The medical device 110 includes a head portion 1120 (also called the implant actuation end). The head portion 1120 is provided with a flanged tip and a hexagonal drive head. However, other components and shapes of the head portion and implant may be used in alternative embodiments. Also visible in FIG. 11 is a bead 1100 of the medical device 110. As disclosed herein, beads of a flexible implant like the bead 1100 are radially expandable. The configuration of beads in the portion of a flexible implant that extends from the head portion 1120 also facilitates flexibility for the implant.

The end plate assembly 105 here includes three different components. Here, any fasteners that might be used have been omitted from this view. The end plate assembly 105 includes the end plate 1105 itself. A tightening head 1110 is configured to screw into an opening in the end plate 1105. As will be discussed in detail below, the tightening head 1110 may be provided with multiple bores in an opening of the tightening head 1110. In this embodiment, the tightening head 1110 has a hexagonal and two cylindrical bores. (Although the two cylindrical bores may be considered a single stepped bore.) Further, the locking head 1115 is also configured to screw into an opening in the end plate 1105. Both the locking head 1115 and the tightening head 1110 can move (rotate and translate) with respect to the end plate 1105. Accordingly, the locking head 1115 and the tightening head 1110 form a nut and bolt type assembly with the end plate 1105 due to the engaging internal and external threads on the components. These components are discussed in greater detail herein.

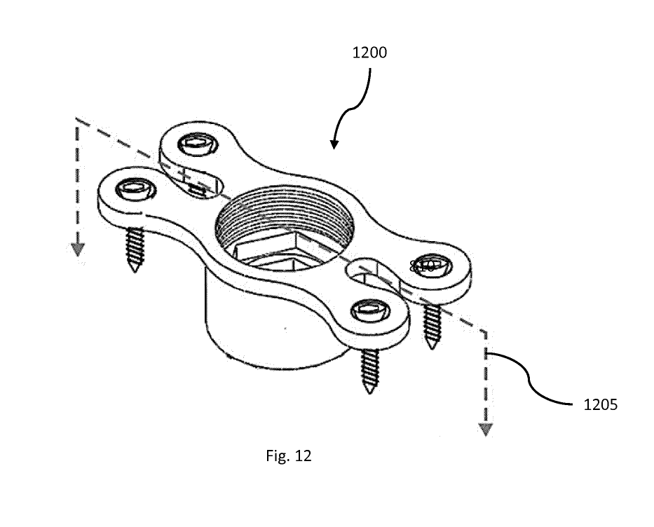

FIG. 12 depicts a perspective view of an end plate assembly 1200 in accordance with an illustrative embodiment. In alternative embodiments, fewer, additional, and/or different components may be included in the system. FIG. 12 shows a perspective view of the end plate assembly 1200. It also shows a section plane 1205. The section plane 1205 demonstrates where a cross-section has been cut in the end plate assembly 1200 for the views that are discussed below in FIGS. 13A, 13B, 16A, 16B, 17A, 17B, 18A, 18B, 19A, 19B, 20A, 20B, 21A, 21B, 22A, and 22B.

FIG. 13A depicts a cross-sectional view of an end plate assembly in accordance with an illustrative embodiment. In alternative embodiments, fewer, additional, and/or different components may be included in the system. FIG. 13B depicts a perspective cross-sectional view of an end plate assembly in accordance with an illustrative embodiment. In alternative embodiments, fewer, additional, and/or different components may be included in the system.

Visible in FIGS. 13A and 13B is the end plate 1105 and the tightening head 1110. The tightening head 1110 is externally threaded and the opening of the end plate 1105 has an internally threaded cylindrical bore 1300 so that the tightening head 1110 can be screwed into the end plate 1105. This interaction can be seen at element 1305.

The tightening head 1110 itself has three different bores (although as will be explained it may be considered two bores with one bore have a step). A first bore 1310 is an internal hexagonal bore. The first bore 1310 may also be considered stepped because a second bore 1315 is smaller than the first bore 1310. The first bore 1310 is configured to fit a tensioning tool so that the tightening head 1110 can be turned within the end plate 1105. Other shapes than a hexagon may be used in alternative embodiments for the shape of the first bore 1310.

The tightening head 1110 also includes the second bore 1315. The second bore 1315 is an internal cylindrical bore designed to accommodate a head portion of a medical device. A third bore 1320 is designed to prevent a head portion of a medical device from passing through the third bore 1320. The second bore 1315 and the third bore 1320 may together be considered a singled stepped cylindrical bore, because the third bore 1320 is smaller than the second bore 1315 and therefore forms a step between the two.

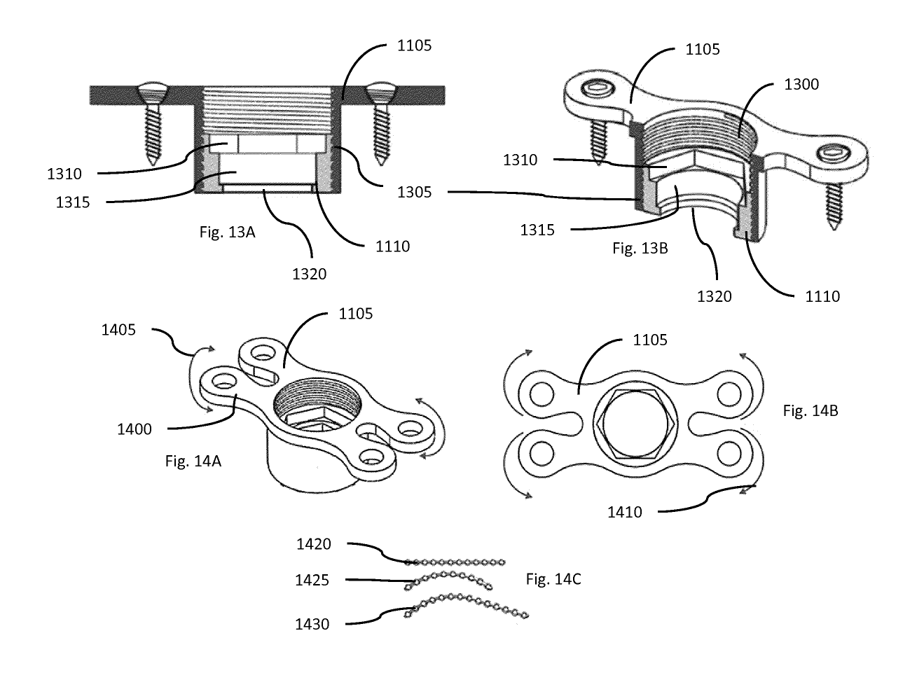

FIG. 14A depicts a perspective view of the adjustability of an end plate 1105 in accordance with an illustrative embodiment. In alternative embodiments, fewer, additional, and/or different components may be included in the system. FIG. 14B depicts a top view of the adjustability of an end plate 1105 in accordance with an illustrative embodiment. In alternative embodiments, fewer, additional, and/or different components may be included in the system.

The end plate 1105 includes fixation portions 1400. In this embodiment, the fixation portions are tab like structures that extend from a second portion of the end plate 1105. The arrows 1405 and 1410 show various directions a fixation portion 1400 might be moved to adjust an end plate 1105 to the contours of the surface of a bone where the end plate 1105 might be mounted. The fixation portion can be bent or adjusted to the contours of the pelvis or any other bone that the embodiments disclosed herein are being used on.

FIG. 14C depicts various shapes of pelvic contours in accordance with an illustrative embodiment. In alternative embodiments, fewer, additional, and/or different components may be included in the system. Contours 1420, 1425, and 1430 show examples of what the surface of a pelvis may be shaped like. Accordingly, the end plate 1105, and particularly the end plate 1105 's fixation portion 1400, may be adjusted to fit or approximate the contours of a bone such as pelvis contours 1420, 1425, and/or 1430.

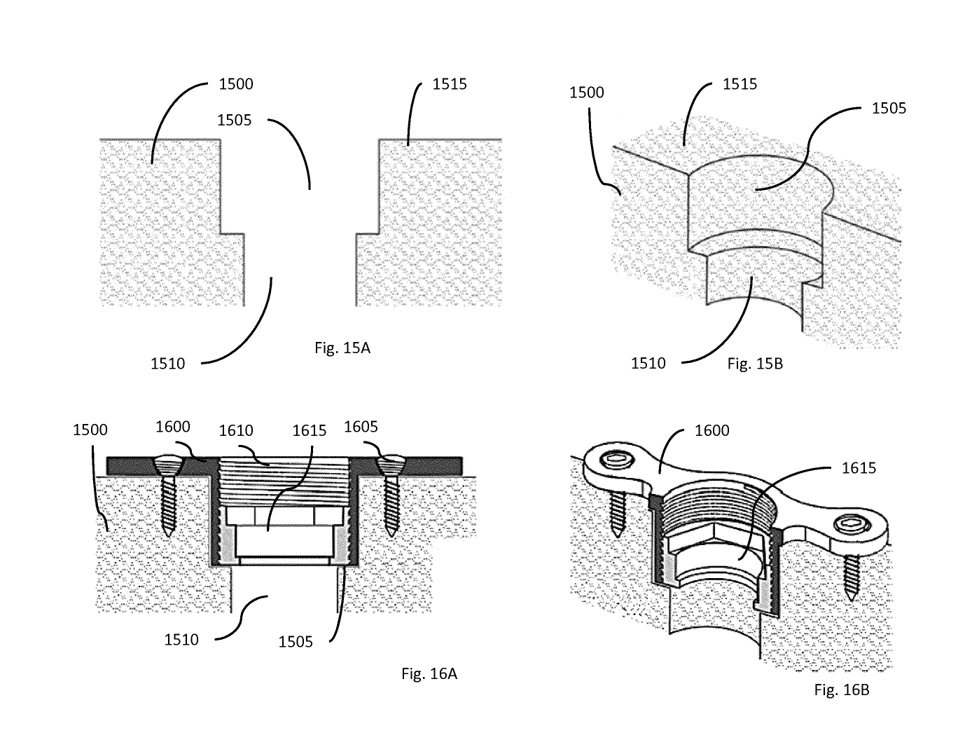

FIG. 15A depicts a cross-sectional view of a stepped bore in a proximal section of a fractured human pelvic bone in accordance with an illustrative embodiment. In alternative embodiments, fewer, additional, and/or different components may be included in the system. FIG. 15B depicts a perspective cross-sectional view of a stepped bore in a proximal section of a fractured human pelvic bone in accordance with an illustrative embodiment. In alternative embodiments, fewer, additional, and/or different components may be included in the system.

In the proximal bone segment 1500 shown in FIGS. 15A and 15B, a bore or bone tunnel has been formed. A first bore section 1505 has been formed at the proximal end, closer to the natural surface 1515 of the bone. The first bore section 1505 is larger (wider diameter and larger area) than a second bore section 1510. Both the first bore section 1505 and the second bore section 1510 are cylindrical in this embodiment. In other embodiments, the shapes of the various bores may be any shape. Together, the first bore section 1505 and the second bore section 1510 form a stepped bore at the proximal end of the bore or bone tunnel. Further, the views in FIGS. 15A and 15B do not show the entire length of the second bore section 1510.

FIG. 16A depicts a cross-sectional view of an end plate assembly in a stepped bore in a proximal section of a fractured human pelvic bone in accordance with an illustrative embodiment. In alternative embodiments, fewer, additional, and/or different components may be included in the system. FIG. 16B depicts a perspective cross-sectional view of an end plate assembly in a stepped bore in a proximal section of a fractured human pelvic bone in accordance with an illustrative embodiment. In alternative embodiments, fewer, additional, and/or different components may be included in the system.

FIGS. 16A and 16B show an end plate 1600 inserted into the first bore section 1505. The second bore section 1510 is too narrow to accommodate the end plate 1600. Further, the fixation portions of the end plate 1600, contact the surface of the proximal bone segment, also preventing further insertion of the end plate. The fixation portions of the end plate 1600 are affixed with fasteners such as cortex screw 1605. The end plate 1600 includes an opening 1610. The end plate 1600 also has within the opening 1610 a tightening head 1615. The end plate 1600 is secured both through the cortex screw fasteners and through an interference fit between the end plate 1600's second portion that is cylindrical an the stepped bone tunnel (or bore).

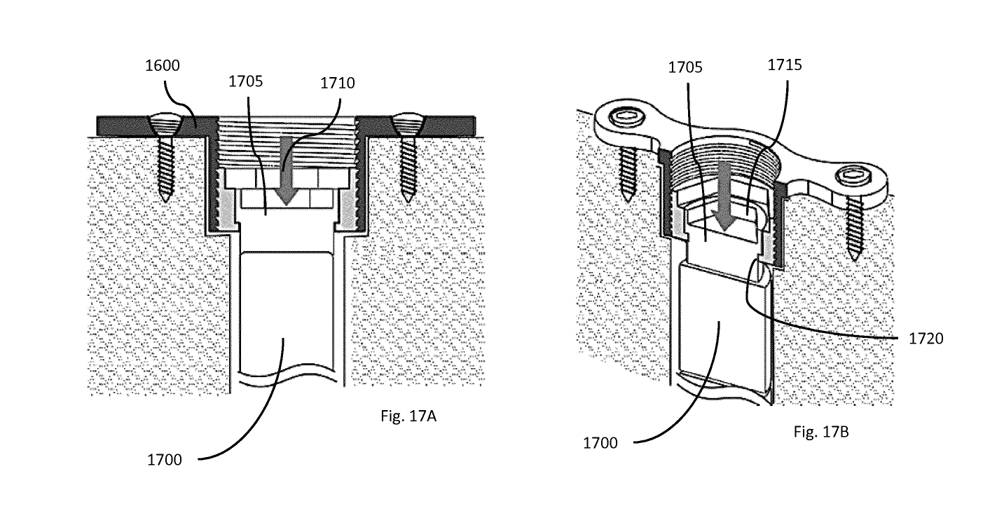

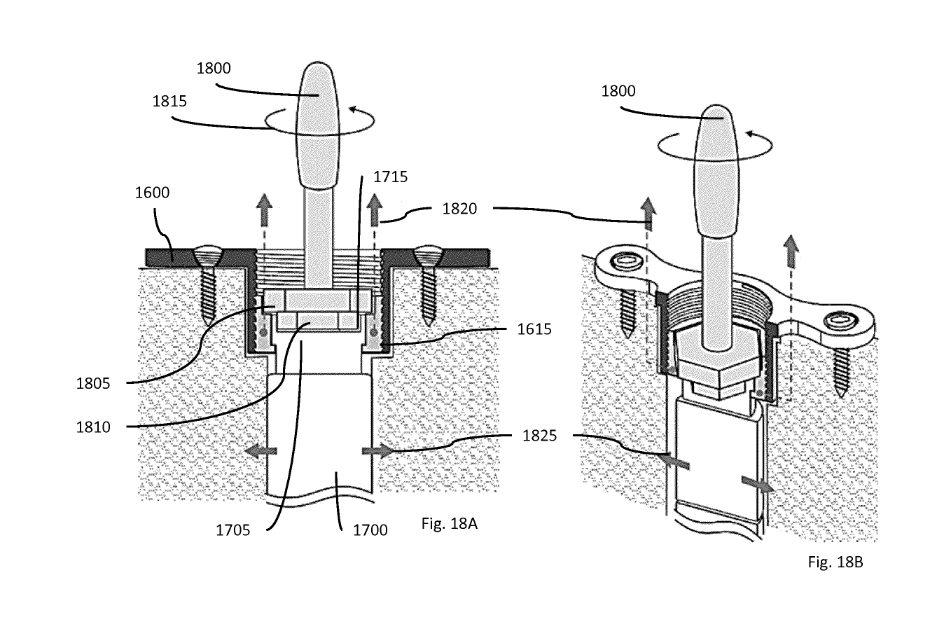

FIG. 17A depicts a cross-sectional view of a medical device 1700 in an end plate assembly in a stepped bore in a proximal section of a fractured human pelvic bone in accordance with an illustrative embodiment. In alternative embodiments, fewer, additional, and/or different components may be included in the system. FIG. 17B depicts a perspective cross-sectional view of a medical device 1700 in an end plate assembly in a stepped bore in a proximal section of a fractured human pelvic bone in accordance with an illustrative embodiment. In alternative embodiments, fewer, additional, and/or different components may be included in the system.

The medical device 1700 has a head portion 1705. The medical device 1700 can be inserted through openings (bores) in the end plate 1600 and the tightening head 1615. The head portion 1705 is shaped so as to not go past a stepped portion 1720 of the tightening head 1615. An arrow 1710 shows the direction in which the medical device is inserted. The head portion 1705 of the medical device 1700 also includes a hexagonal inset 1715, which can accommodate a tensioning tool so that the medical device 1700 can be adjusted, tensioned, etc. In alternative embodiments, the inset may be other shapes to accommodate differently shaped tensioning tools, such as triangular, square, etc. shaped tensioning tools. In some embodiments, a single inset may be shaped/configured to accommodate more than one shape of tensioning tool.

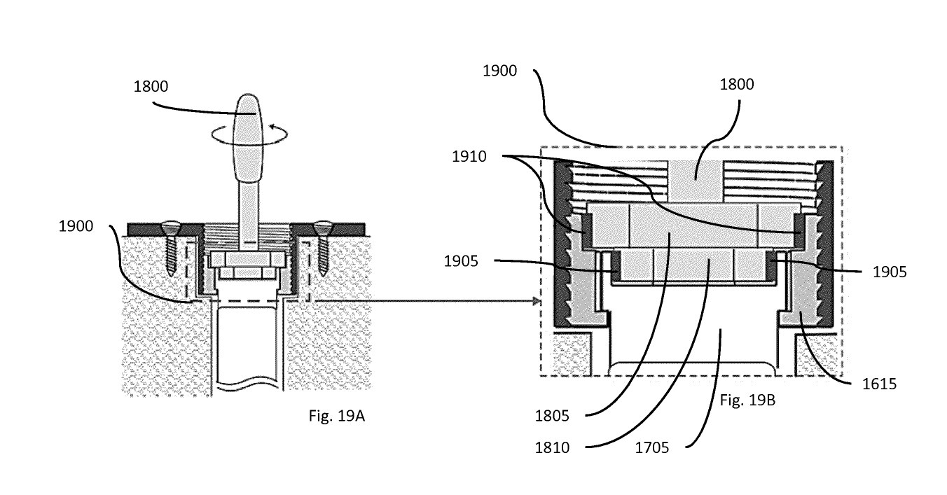

FIG. 18A depicts a cross-sectional view of a tensioning tool 1800 used to adjust a medical device 1700 in a fractured human pelvic bone in accordance with an illustrative embodiment. In alternative embodiments, fewer, additional, and/or different components may be included in the system. FIG. 18B depicts a perspective cross-sectional view of a tensioning tool 1800 used to adjust a medical device 1700 in a fractured human pelvic bone in accordance with an illustrative embodiment. In alternative embodiments, fewer, additional, and/or different components may be included in the system.

The tensioning tool 1800 can be rotated in the direction 1815 in order to actuate the medical device 1700 and engage the hexagonal bore of the tightening head 1615. Portion 1810 of the tensioning tool 1800 engages the hexagonal drive head in the head portion 1705 of the medical device 1700. The larger portion 1805 engages the hexagonal bore of the tightening head 1615.

When the tensioning tool 1800 is turned, simultaneous rotation input is applied to the medical device 1700 and the tightening head 1615. Because of the thread based engagement between the tightening head 1615 and the end plate 1600, the tightening head 1615 acts as a nut (in part because the end plate 1600 is affixed to the bone) and translates, pulling the distal bone segment via the anchored medical device 1700 toward the proximal bone segment, thus compressing the proximal and distal bone segments against each other. Arrows 1820 indicate how the tightening head 1615 engages with the end plate 1600 to move away from the fracture and subsequently compress the distal and proximal bone segments.

The compression of the fracture as just described happens simultaneously (when the tensioning tool 1800 is used) with the actuation of of the medical device 1700. The rotational input in direction 1815 applied to the head portion 1705 of the medical device 1700 causes the medical device 1700 to actuate (expand as indicated by arrows 1825).

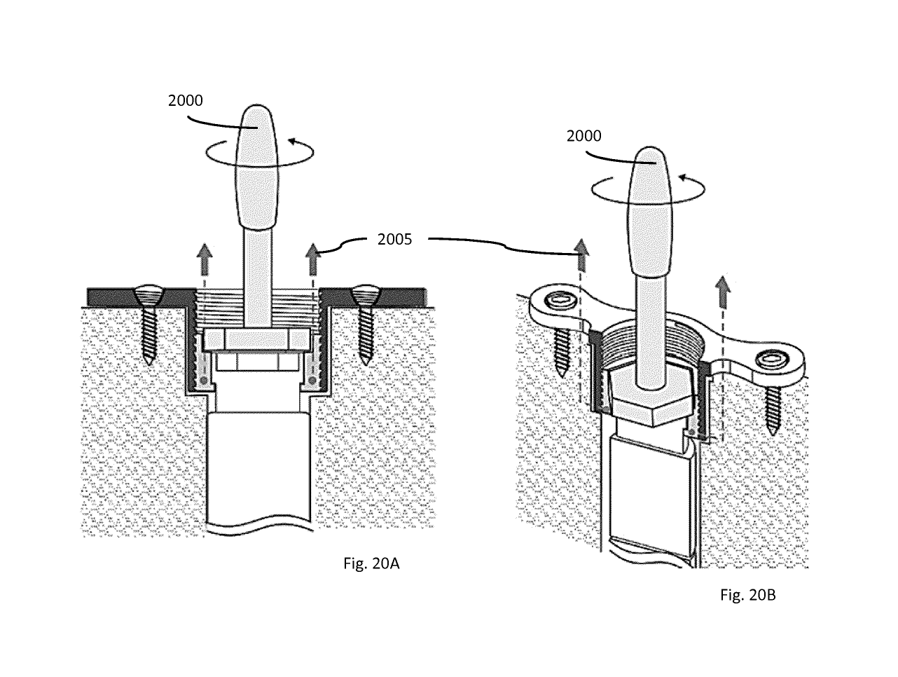

FIG. 19A depicts a detail area of a cross-sectional view of a tensioning tool used to adjust a medical device in a fractured human pelvic bone in accordance with an illustrative embodiment. In alternative embodiments, fewer, additional, and/or different components may be included in the system. FIG. 19B depicts an enlarged cross-sectional view of a tensioning tool used to adjust a medical device in a fractured human pelvic bone in accordance with an illustrative embodiment. In alternative embodiments, fewer, additional, and/or different components may be included in the system.

FIGS. 19A and 19B show in greater detail 1900 the simultaneous engagement of the head portion 1705 of the medical device 1700 and the tightening head 1615. The tightening head 1615 is engaged, for example, at points 1910 by the portion 1805 of the tensioning tool 1800. The head portion 1705 of the medical device 1700 is engaged, for example, at points 1905 by portion 1810 of the tensioning tool 1800.

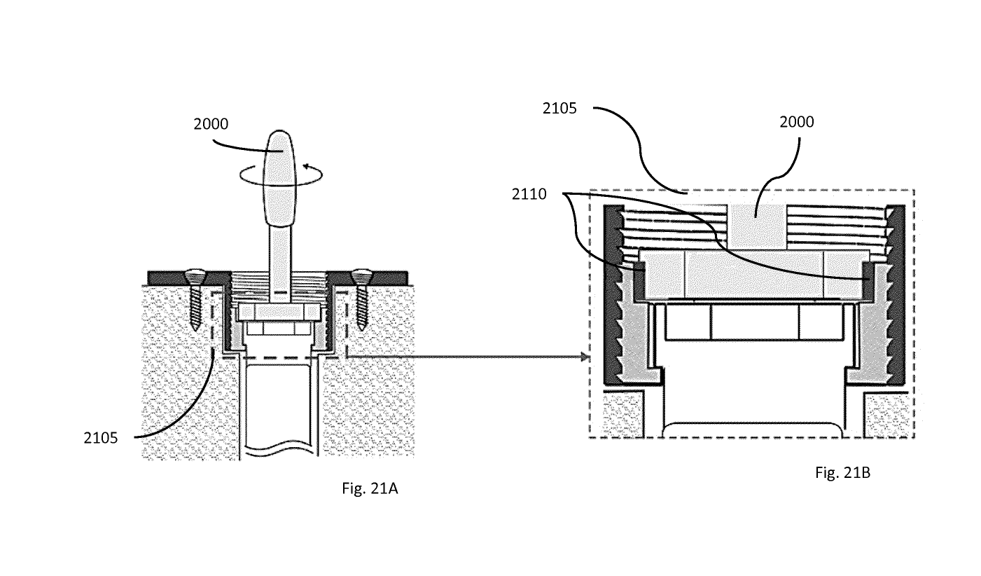

FIG. 20A depicts a cross-sectional view of a tensioning tool 2000 used to adjust a tightening head of an end plate assembly in accordance with an illustrative embodiment. In alternative embodiments, fewer, additional, and/or different components may be included in the system. FIG. 20B depicts a perspective cross-sectional view of a tensioning tool 2000 used to adjust a tightening head of an end plate assembly in accordance with an illustrative embodiment. In alternative embodiments, fewer, additional, and/or different components may be included in the system.

If further tensioning of the medical device is desired, a tensioning tool 2000 may be utilized. The tensioning tool 2000 engages the tightening head to ultimately compress the proximal and distal bone segments at the fracture, but does not engage the head portion of the medical device, as evidenced by arrows 2005. In this way, more precise fracture reduction can be achieved. Final control over bone compression forces can also be exacted. Degrees of primary and secondary tensioning can be decided by a surgeon on a case by case basis. Furthermore, tensioning with the tensioning tool 2000 can further tension a medical device without causing additional radial expansion of the medical device. In an alternative embodiment, a tensioning tool similar to the tensioning tool 2000 may be used, except the tensioning tool would engage the head portion of a medical device but not the tightening head.

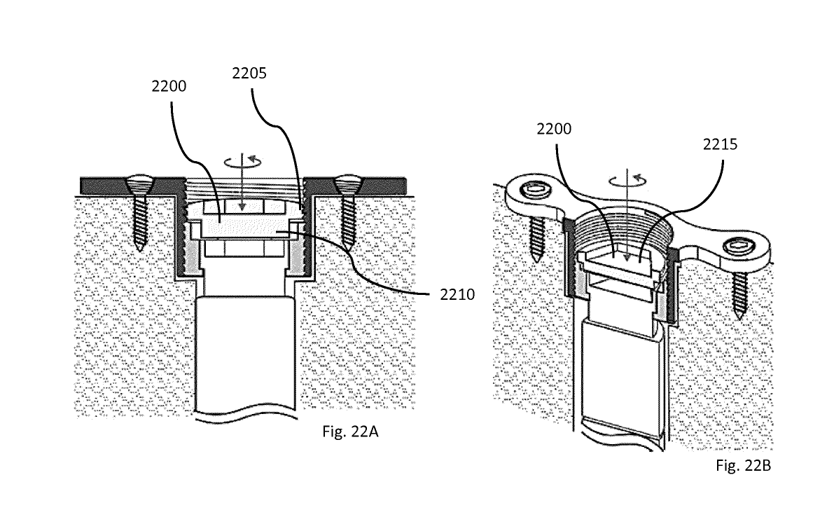

FIG. 21A depicts a detail area of a cross-sectional view of a tensioning tool 2000 used to adjust a tightening head of an end plate assembly in accordance with an illustrative embodiment. In alternative embodiments, fewer, additional, and/or different components may be included in the system. FIG. 21B depicts an enlarged cross-sectional view of a tensioning tool 2000 used to adjust a tightening head of an end plate assembly in accordance with an illustrative embodiment. In alternative embodiments, fewer, additional, and/or different components may be included in the system.

FIGS. 21A and 21B show in greater detail 2105 the engagement of the tightening head 1615. The tightening head 1615 is engaged, for example, at points 2110. The head portion 1705 of the medical device 1700 is not engaged in this embodiment.

FIG. 22A depicts a cross-sectional view of locking head 2200 in an end plate assembly in accordance with an illustrative embodiment. In alternative embodiments, fewer, additional, and/or different components may be included in the system. FIG. 22B depicts a perspective cross-sectional view of locking head 2200 in an end plate assembly in accordance with an illustrative embodiment. In alternative embodiments, fewer, additional, and/or different components may be included in the system.

The locking head 2200 has an outer thread 2205 which engages the opening of the end plate so that the locking head 2200 can be screwed down into the opening. The locking head 2200 also can secure the tightening head to prevent back out of the medical device during biomechanical loading.

Materials for the various components (end plates, tightening heads, locking, heads, fasteners, etc.) may be made of bio-compatible alloys/composites that are used on orthopaedic implants. For example, such materials could be titanium and/or stainless steel alloys. Titanium offers excellent malleability, which would allow for an end plate (including its fixation portion) to be bent easily to adapt to pelvis (or other bone) contours. Titanium also offers good visualization under imaging techniques so future doctors, security, etc. can see precisely what is happening with an implant. Stainless steel is also common, well accepted, safe, strong and economical. Stainless steel is often used for fixation devices.

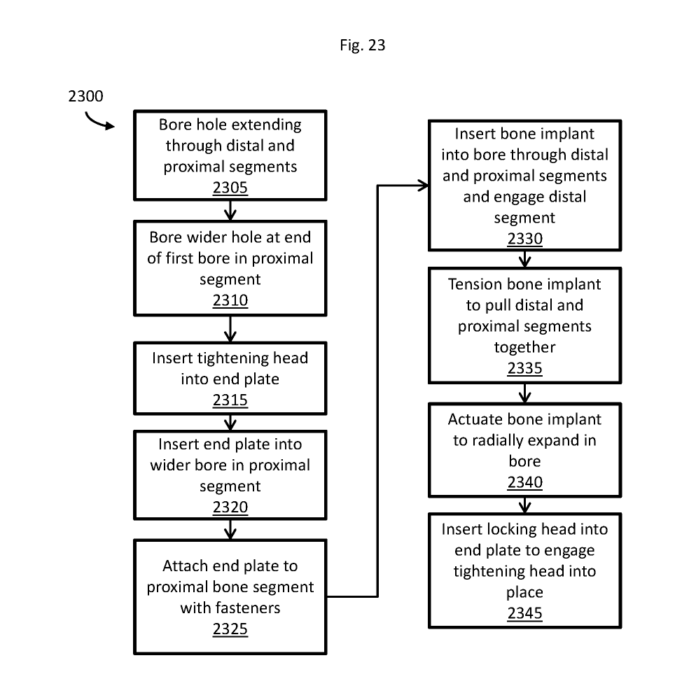

FIG. 23 is a flow diagram illustrating a method 2300 for inserting and adjusting a medical device in accordance with an illustrative embodiment. In alternative embodiments, fewer, additional, and/or different operations may be performed. Also, the use of a flow diagram is not meant to be limiting with respect to the order of operations performed. In an operation 2305, a first hole (or bone tunnel) is bored extending through distal and proximal bone segments across a fracture.

In an operation 2310, a second hole (or bone tunnel) wider than the first hole and concentric with the first hole is bored at the proximal end of the first hole (and in the proximal bone segment). This results in the stepped bone tunnel desired for an end plate as disclosed herein. In an operation 2315, a tightening head is inserted into an end plate. In an operation 2320, the end plate is inserted into the wider second hole (bore) in the proximal bone segment.

In an operation 2325, the end plate is attached to the proximal bone segment with fasteners. In an operation 2330, a bone implant is inserted into the bore (or bone tunnel) and the implant extends through the proximal bone segment, across a fracture, and into the distal bone segment. The bone implant also engages the distal bone segment, such as through threads that anchor the bone implant to the distal bone segment.

In an operation 2335, the bone implant is tensioned to pull the proximal bone segment and the distal bone segment together, which compresses the two segments at the fracture. In an operation 2340, the bone implant is actuated to radially expand in the bore (or bone tunnel). In an operation 2345, a locking head is inserted into the end plate to engage the tightening head and/or the bone implant to ensure that the tightening head and/or the bone implant stay in place.

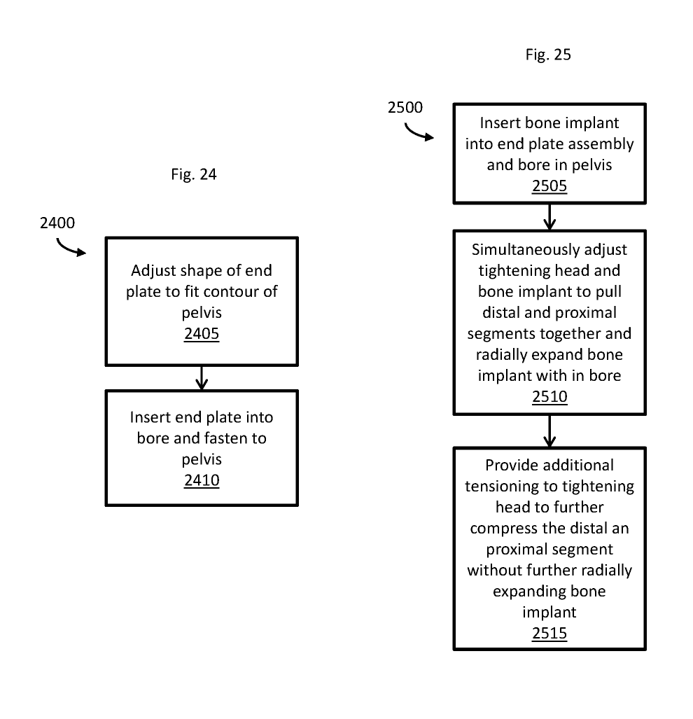

FIG. 24 is a flow diagram illustrating a method 2400 for adjusting an end plate in accordance with an illustrative embodiment. In alternative embodiments, fewer, additional, and/or different operations may be performed. Also, the use of a flow diagram is not meant to be limiting with respect to the order of operations performed.

In an operation 2405, the shape of an end plate to fit the contour of a pelvis. In alternative embodiments, the fractured bone may not be a pelvis. In such embodiments, the shape of the end plate may still similarly be reshaped to fit the contour of whatever is in fact fractured and utilizing the end plate as disclosed herein. In an operation 2410, the end plate is inserted into a bore and fastened to the pelvis as disclosed herein.

FIG. 25 is a flow diagram illustrating a method 2500 for simultaneously adjusting the radial expansion of a bone implant and pulling a distal and proximal bone segment together in accordance with an illustrative embodiment. In alternative embodiments, fewer, additional, and/or different operations may be performed. Also, the use of a flow diagram is not meant to be limiting with respect to the order of operations performed.

In an operation 2505, a bone implant is inserted into an end plate assembly and bore in a pelvis. In alternative embodiments, the bone may not be a pelvis. In an operation 2510, the tightening head and the bone implant are simultaneously adjusted with a particular tensioning tool that engages both at the same time. This pulls the distal bone segment toward the proximal bone segment and radially expands and stiffens the bone implant at the same time. In an operation 2515, a surgeon can optionally provide additional tensioning to the tightening head to further compress the distal and proximal bone segments as needed. This tensioning can be done without further radially expanding the bone implant itself.

The methods and systems disclosed herein may be utilized with a variety of patients that have varying degrees of osteoporosis, or no osteoporosis at all. The embodiments disclosed herein provide for great variability and adjustment so that the embodiments are practical for a wide patient population.

The methods and systems disclosed herein can also be adapted to many other implants other than a flexible pelvic implant. Such other implants may also fixate to weaker cancellous bone via a pre-drilled channel and utilizing an end plate assembly as disclosed herein can allow for improved stress distribution as disclosed herein.

An advantage for surgeons of the embodiments disclosed herein is that the adjustment and tensioning provides a tangible feel for the degree of bone impaction. Such hands on interaction may increase effectiveness of a surgeon utilizing the various embodiments disclosed herein.

As discussed herein, there are many advantages to utilizing the systems and methods disclosed herein. Stress can be distributed over increased bone-implant interface. Due to the wider buttressing, a bone-implant interface is increased spreading forces over an increased surface area of stronger cortical bone, thus preventing undue stress concentration.

Another advantage of the embodiments disclosed herein is reduced strain on an osteoporotic bone. Load transmitted at the bone-implant interface is distributed over a larger surface area using the embodiments herein. Utilizing the added surface area of the stress shielding end plate reduces the strain on the osteoporotic bones, which thereby reduces the occurrence of micro fracture and bone resorption.

The systems and methods disclosed herein also have the advantage of variable tensioning, which can be customized based on the degree of osteoporosis in a bone. Variable tensioning allows the surgeon to exercise control over the extent of tensioning and hence extent of bone compaction for a fracture. Such adjustments can be made by the surgeon based on bone state (e.g., the degree of the osteoporosis) and fracture specifics (e.g., location, type, extent of precise reduction required).

The systems and methods disclosed herein also provide improved torsional stability and resistance to implant backout. An end plate fixation as disclosed herein contacts stronger cortical bone. Further, a fixated end plate helps lock in place a bone implant, providing added torsional stability and resistance to backout.

The systems and methods disclosed herein also provide enhanced fracture management. The embodiments disclose herein ensure effective and prolonged impact of fractured bone segments. This is a key element in the surgical management of osteoporotic fractures and reduces the risk of implant failure. All of this helps achieve predictable and timely results for the healing of a fracture interface.

The systems and methods disclosed herein also provide the advantage of being adaptable to diverse implant constructions. The embodiments herein (particularly the end plate assembly) can be fit and adapted to any flexible/rigid implant construction, and does not depend on the working mechanism of an implant for its performance. Moreover, it can be of use with other fracture repair implants that use variable impaction of bone segments, especially those that deal with osteoporotic bones. In various embodiments, the size of the construction of various aspects of the embodiments disclosed herein may be scaled up and/or miniaturized based on applications to different bone structures.

Bone impaction is important in the surgical management of osteoporotic fractures as it reduces the risk of implant failure. Controlled impaction can be attained by tensioning internal fixation devices. Fracture compression increases the contact area across the fracture and increases stability of the fracture. It also decreases the fracture gap and decreases stress on the orthopaedic implant. This compression can be static, where the compression is produced by the fixation device alone, or dynamic, where body weight or muscle forces are used to produce additional compression. This is beneficial to fracture healing because it improves stability, opening the possibility for primary bone healing with minimal callus formation.

In the case of osteoporotic bones, if the load transmitted at the bone-implant interface exceeds the strain tolerance of osteoporotic bone, microfracture and resorption of bone with loosening of the implant and secondary failure of fixation can occur. A common mode of failure of internal fixation in osteoporotic bone is bone failure (especially when dominant mechanical characteristic of osteoporotic bone is low density and reduced strength characteristics) rather than implant breakage. Because attachment to bone, by a screw for example, is directly related to bone density, several strategies can be used when osteoporotic bone is encountered. Hence, specific strategies to augment fixation strength in osteoporotic bone are disclosed herein.

One or more flow diagrams may have been used herein. The use of flow diagrams is not meant to be limiting with respect to the order of operations performed. The herein described subject matter sometimes illustrates different components contained within, or connected with, different other components. It is to be understood that such depicted architectures are merely illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. An apparatus comprising:
an end plate comprising:
a fixation portion configured to attach to a proximal bone segment; and
a second portion comprising an opening configured to receive a medical device; and
a tightening head shaped for selective placement within the opening, wherein the tightening head is further configured for engagement with the medical device.

2. The apparatus of claim 1, wherein the tightening head comprises:
an outer portion, wherein the outer portion comprises a first threaded portion; and
an inner portion comprising:
a first bore at a top of the inner portion,
a second bore at a middle of the inner portion, and
a third bore at a bottom of the inner portion, wherein the first bore, the second bore, and the third bore are different sizes.

3. The apparatus of claim 2, wherein the second portion of the end plate further comprises a second threaded portion that is configured to engage the first threaded portion of the tightening head.

4. The apparatus of claim 3, wherein the medical device comprises a head portion and an extended portion that extends from the head portion, wherein the head portion has a larger diameter than the third bore, wherein the second bore has a shape that corresponds to the head portion of the medical device such that the second bore holds the head portion, and wherein the extended portion of the medical device comprises a third threaded portion that is configured to engage the distal bone segment.

5. The apparatus of claim 4, wherein the medical device comprises a flexible adjustable bone implant, wherein the head portion is configured to rotate independent of the extended portion, and further wherein the extended portion is configured to change width in response to rotational movement of the head portion.

6. The apparatus of claim 2, wherein the area of the first bore is larger than the area of the second bore, and further wherein the area of the second bore is larger than the area of the third bore.

7. The apparatus of claim 6, wherein the first bore comprises a hexagonal stepped bore, wherein the second bore comprises a cylindrical stepped bore, and wherein the first bore, the second bore, and the third bore form a hollow cavity in the tightening head.

8. The apparatus of claim 1, further comprising a locking head configured to prevent the tightening head from rotating within the end plate.

9. The apparatus of claim 8, wherein:
the locking head comprises a fourth threaded portion and a non-threaded portion;
the fourth threaded portion of the locking head is configured to engage the second threaded portion of the end plate; and
the non-threaded portion of the locking head is shaped to fit in the first bore of the tightening head.

10. A method of compacting a bone fracture, the method comprising:
inserting an adjustment portion of an end plate into a bone tunnel, wherein the adjustment portion is configured to receive a medical device;
attaching a fixation portion of the end plate to a proximal bone segment;
inserting the medical device into the bone tunnel and the adjustment portion of the end plate such that a head portion of the medical device engages a tightening head in the adjustment portion; and
adjusting the tightening head causing the medical device to be adjusted.

11. The method of claim 10, wherein adjusting the tightening head comprises rotating the tightening head such that the end plate and the medical device pull a distal bone segment closer to the proximal bone segment.

12. The method of claim 11, wherein rotating the tightening head tensions the medical device and moves the head portion toward a top of the adjustment portion of the end plate from which the fixation portion extends.

13. The method of claim 10, further comprising receiving the adjustment portion of the end plate in a step portion of the bone tunnel.

14. The method of claim 10, wherein the bone tunnel extends through at least a portion of the proximal bone segment and at least a portion of a distal bone segment, and further wherein the bone tunnel extends across a fracture between the distal bone segment and the proximal bone segment.

15. The method of claim 10, further comprising rotating the head portion of the medical device such that the medical device changes width in response to rotational movement of the head portion.

16. The method of claim 15, wherein the medical device further comprises a body portion that extends from the head portion and a tail portion at the end of the body portion opposite the head portion, the method further comprising securing the tail portion of the medical device to the distal bone segment, wherein the tail portion of the medical device is threaded to secure the tail portion to the distal bone segment.

17. The method of claim 15, wherein the diameter of the bone tunnel is smaller in a distal bone segment than the diameter of the bone tunnel in the proximal bone segment, the method further comprising securing the medical device to the distal bone segment by adjusting the medical device such that the width of the bone implant increases.

18. The method of claim 10, further comprising
inserting a tensioning tool into the adjustment portion and the tightening head;
rotating the tensioning tool, wherein the tensioning tool is configured to move the tightening head vertically within the adjustment portion; and
inserting a locking head, wherein the locking head prevents rotation of the head portion of the medical device and the tightening head.

19. The method of claim 10, wherein the fixation portion of the end plate comprises a plurality of arms, the method further comprising fastening the plurality of arms to the proximal bone segment.

20. An system comprising:
a bone implant;
an end plate comprising
a fixation portion configured to attach to a proximal bone segment and
a second portion comprising an opening configured to receive the bone implant; and
a tightening head shaped for selective placement within the opening, wherein the tightening head is further configured for engagement with the bone implant, and further wherein the tightening head is configured to engage the end plate and the bone implant.
